(19) United States
(12) Patent Application Publication
(10) Pub. No.: US 2010/0166872 A1
(43) Pub. Date: Jul. 1, 2010

(54) NOVEL IMPROVED COMPOSITIONS FOR CANCER THERAPY

(76) Inventors: Amarjit Singh, Mumbai (IN); Singh Sarabjit, Chandigarh (IN); Ajay K. Gupta, Delhi (IN); Mangesh M. Kulkarni, Thane (IN)

Correspondence Address: LADAS & PARRY LLP, 26 WEST 61ST STREET, NEW YORK, NY, 10023, US

(21) Appl. No.: 12/083,594

(22) PCT Filed: Oct. 19, 2006
(86) PCT No.: PCT/IN2006/000427

§ 371 Date: Apr. 14, 2008

(30) Foreign Application Priority Data
Oct. 21, 2005 (IN) 1327/MUM/2005
Jul. 10, 2006 (IN) 1081/MUM/2006

(51) Int. Cl. A61K 31/337; A61K 9/14; A61P 35/00; A61K 38/20; A61K 38/21; A61K 39/395

(52) U.S. Cl. 424/499; 424/501; 424/502; 514/449; 424/85.2; 424/85.4; 424/141.1; 424/178.1; 977/773; 977/915

(57) ABSTRACT

The present invention relates to novel and improved compositions of anticancer drugs, preferably taxanes, such as paclitaxel and docetaxel, their derivatives or their analogues, methods of manufacturing these compositions and methods of fractionating the particles in particular size range and methods of treating cancer patients with these compositions, which provide reduced chemotherapy-induced side-effects especially reduced chemotherapy-induced-alopecia. The composition is such that there is substantially no free drug in the said composition.

The present invention relates to novel and improved compositions of anticancer drugs. It relates to novel and improved compositions for cancer therapy having substantially reduced chemotherapy-induced side-effects.

The present invention relates to novel and improved compositions of anticancer drugs including but not limited to alkylating agents, antimetabolites, antibiotic anticancer agents, plant alkaloids, anthracenediones, natural products, hormones, hormone antagonists, miscellaneous agents, radiosensitizers, platinum coordination complexes, adrenocortical suppressants, immunosuppressive agent, functional therapeutic agents, gene therapeutic agent, antisense therapeutic agent, tyrosine kinase inhibitor, monoclonal antibody, immunotoxin, radioimmunoconjugate, cancer vaccine, interferon, interleukin, substituted ureas, taxanes and COX-2 inhibitors.

The present invention relates to novel and improved compositions of anticancer drugs, preferably Taxanes, such as paclitaxel and docetaxel, their derivatives or their analogues, methods of manufacturing these formulations and methods of treating cancer patients with these compositions.

The present invention relates to novel and improved compositions of anticancer drugs, preferably Taxanes, such as paclitaxel and docetaxel, their derivatives or their analogues, methods of manufacturing these compositions and methods of fractionating the particles in particular size range and methods of treating cancer patients with these compositions, which provide reduced chemotherapy-induced side-effects especially reduced chemotherapy-induced-alopecia. The composition is such that there is substantially no free drug in the said composition.

The novel and improved compositions of anti-cancer drugs, preferably Taxanes such as paclitaxel and docetaxel, their derivatives or their analogues, are colloidal delivery systems, for cancer therapy with drastically reduced chemotherapy-induced-alopecia, prepared in a defined size range, with substantially no free drug present in the composition.

### BACKGROUND OF THE INVENTION

A wide variety of anticancer agents have been developed till date for treatment of various types of cancers in mammals and newer and newer agents are being developed as chemotherapeutics wherein the research is aimed at developing tumor specific anti-cancer agents while increasing the potency against drug-resistant tumors. Further newer clinical protocols involve combining anti-cancer drugs to produce increased therapeutic efficacy. Such newer discoveries are on-going, but to-date chemotherapeutic agents such as 5-Fluorouracil (5FU), Doxorubicin and the Taxanes are a mainstay of therapy for patients with a variety of cancers including ovarian, breast, lung, colon, prostate, head and neck, cervical and brain and others.

However the use of these and other drugs have been limited by associated toxicities, including nausea, myelosuppression, alopecia, vomiting and stomatitis and also cardio-toxicity.

From amongst all these associated toxicities mentioned above, alopecia (or hair loss) due to chemotherapy is one of the most distressing and traumatic side-effect for cancer patients as it causes depression, loss of self-confidence, and humiliation in men and women of all ages. Some patients refuse to undergo treatment because of the physical and emotional angst that results from treatment-related alopecia. Hair loss has a significant influence upon patient's psychological condition and it is a serious problem affecting the quality of life of patient's. There is thus a pressing need to provide a type of cancer treatment with drastically reduced chemotherapy-induced-alopecia.

Taxanes are anticancer cytotoxics that stabilize cellular microtubules. Taxane compounds useful in the composition and methods described herein include paclitaxel and docetaxel, as well as natural and synthetic analogs thereof, which possess anticancer or anti-angiogenic activity. Paclitaxel and Docetaxel have substantial activity, and one or both of these agents are widely accepted as components of therapy for advanced breast, lung, and ovarian carcinomas.

Docetaxel is an antineoplastic agent belonging to the taxoid family. It is prepared by semi-synthesis, beginning with a precursor extracted from the renewable needle biomass of yew plants. Taxotere® is sterile docetaxel injection concentrate, available in single-dose vials containing docetaxel and polysorbate 80, to be administered intravenously after diluting with a diluent like ethanol in water for injection and is indicated for the treatment of patients with locally advanced or metastatic breast cancer after failure of prior chemotherapy. TAXOTERE in combination with doxorubicin and cyclophosphamide is indicated for the adjuvant treatment of patients with operable node-positive breast cancer.

Paclitaxel, belonging to the taxane class of chemotherapy agents has been widely used for many years in intravenous forms for the treatment of breast and ovarian cancer or non-small cell lung carcinoma (NSCLC). Along with the tremendous potential that paclitaxel has shown as an antitumor drug, clinical problems with solubility, toxicity, poor bioavailability and development of drug resistance are sufficiently severe that the need for formulations of paclitaxel derivatives or analogues with better therapeutic efficacy and less toxicity is very clear.

Paclitaxel (Taxol®) is available as a solution for i.v. infusion in a vehicle composed of Cremophor® EL that has been shown to cause toxic effects such as life-threatening anaphylaxis. This Cremophor/Ethanol formulation of paclitaxel precipitates upon dilution with infusion fluid, and fibrous precipitates are formed in some compositions during storage for extended periods of time. Additional information regarding Cremophor formulations of paclitaxel may be found in Agharkar et al., U.S. Pat. No. 5,504,102.

Recently introduced Abraxane®, is protein-bound paclitaxel particles for injectable suspension. It is an albumin-bound form of paclitaxel which breaks quickly in the liver to release free drug which then circulates in the blood to produce the initial therapeutic response, however it also manifests toxic side effects, such as complete hair loss, infections due to low WBC count, fatigue, weakness and inflammation etc. Complete hair loss, or alopecia, almost always occurs with these dosage forms of Paclitaxel. This usually involves the loss of eyebrows, eyelashes, and pubic hair, as well as scalp hair.

A number of U.S. patent Numbers are listed against this product Abraxane®, these include, U.S. Pat. Nos. 5,439,686; 5,498,421; 5,560,933; 5,665,382; 6,096,331; 6,506,405; 6,537,579; 6,749,868 and 6,753,006.

In accordance with the invention in the above mentioned patents there are provided compositions and methods useful for the in vivo delivery of substantially water insoluble pharmacologically active agents (such as the anticancer drug paclitaxel) in which the active agent is delivered in the form of suspended particles associated or coated with protein (which acts as a stabilizing agent). In these inventions attempt has been made to provide an improvised drug protein microspheres to deliver substantially water insoluble active agents in aqueous suspensions for parenteral administration that does not cause allergic reactions caused due to the presence of emulsifiers and solubilizing agents like Cremophor employed in Taxol.

In U.S. Pat. No. 5,439,686 the inventors have discovered that substantially water insoluble pharmacologically active agents can be delivered in the form of microparticles that are suitable for parenteral administration in aqueous suspension. The invention compositions comprise substantially water insoluble active agents (as a solid or liquid) contained within a polymeric shell, the polymeric shell being a biocompatible polymer crosslinked by the presence of disulfide bonds.

U.S. Pat. No. 5,560,933 claims a method of preparation for the above mentioned composition of their invention, it claims “A method for the preparation of a substantially water insoluble pharmacologically active agent for in vivo delivery, said method comprising subjecting a mixture comprising: a dispersing agent containing said pharmacologically active agent dispersed therein, and aqueous medium containing a biocompatible polymer capable of being crosslinked by disulfide bonds to sonication conditions for a time sufficient to promote crosslinking of said biocompatible polymer by disulfide bonds to produce a polymeric shell containing the pharmacologically active agent therein”.

U.S. Pat. No. 6,506,405 claims formulation of paclitaxel for treatment of primary tumors in a subject, which achieves high local concentration of said paclitaxel at the tumor site, the formulation being substantially free of cremophor. According to '405 inventors, their formulations which contain albumin and is free of cremophor, shows reduced cerebral or neurologic toxicity than the commercially available Taxol composition that contains cremophor.

U.S. Pat. No. 6,749,868 provides a drug delivery system in which part of the molecules of pharmacologically active agent is bound to the protein (eg. human serum albumin) and is therefore immediately bioavailable upon administration to a mammal and the other part of the pharmacologically active agent is contained within nanoparticles coated by protein. The protein coated drug nanoparticles are prepared by using high shear in the absence of conventional surfactants to yield particles with a diameter of less than about 1 micron, which is then sterile-filtered to provide sterile solid formulations useful for intravenous injection.

In the above patents related to Abraxane®, there is provided a method for the administration of paclitaxel coated with protein (like albumin), wherein the said protein coating also has free protein associated within, such that a portion of the active agent is contained within the protein coating and a portion of the active agent is associated with free protein to be available immediately upon administration. The average diameter of the said particles described in the said prior art inventions is no greater than about 1 micron, wherein the composition comprises of particles ranging in size between 10-200 nm, specifically obtained as these small size particles can be sterile-filtered through a 0.22 micron filter. Basically by the use of albumin bound drug particles (albumin being a biocompatible material), the inventors have suggested reduction in toxicities like myelosuppression and/or neurotoxicity of a pharmacologically active agent like paclitaxel in comparison to the already available Taxol, which comprises of cremophor and is associated with allergic reactions and other toxicities.

But none of the above patents describe or provide a method of manufacturing a paclitaxel composition wherein the composition is in a specific narrow size range and has substantially no free drug, so as to provide a cancer therapy with drastically reduced chemotherapy-induced alopecia, which is one of the most traumatic side-effects for cancer patients. The above patents which are related to the commercially available product Abraxane® provides a product which avoids causing allergic reactions by avoiding emulsifiers like Cremophor, and provides a stable, sterilized microparticular or nanoparticular delivery systems for the substantially water insoluble active agent like paclitaxel, but it fails to provide a formulation of paclitaxel devoid of or having reduced side-effects like alopecia or hair loss. The product leaflet for Abraxane® mentions under PATIENT INFORMATION, Hair loss as one of the important side-effects observed in studies of patients taking Abraxane®. It mentions Complete Hair Loss, or Alopecia, almost always occurs with Abraxane®.

There is a research paper publication related to study of temperature- and pH-sensitive core-shell nanoparticles of paclitaxel for intracellular delivery by Yang et al, Front Biosci. 2005 Sep. 1; 10:3058-67, which describe, encapsulating paclitaxel with temperature and pH-sensitive amphiphilic polymeric poly(N-isopropylacrylamide-co-acrylic acid-co-cholesteryl acrylate) (P(NIPAAm-co-AA-co-CHA)) to form nanoparticles. This research paper however does not discuss or mention the methods of manufacturing these particle compositions and fractionating the particles in a particular specific size range and with substantially no free drug, such that it is suitable to provide a composition with drastically reduced chemotherapy-induced alopecia in cancer patients.

U.S. Pat. No. 5,399,363 relates to surface modified anticancer nanoparticles, wherein the particles consists essentially of a crystalline anticancer agent having a surface modifier preferably which are nonionic and anionic surfactant adsorbed on the surface to maintain as effective average particle size of less than about 1000 nm. The use of surfactants would itself contribute towards the toxicity of the composition. The use of specific range of particle size of paclitaxel nanoparticles composition containing biodegradable polymers so as to achieve reduction in specific chemotherapy-induced side-effects like reduced alopecia is neither demonstrated nor predicted from '363 invention. The specific invention of '363 is to have non-crosslinked surface modifiers absorbed on the surface of crystalline anti-cancer medicaments

U.S. Pat. No. 6,136,846 claims a composition for delivering paclitaxel in vivo comprising paclitaxel, a solvent like ethanol or propylene glycol and a water-miscible solubilizer like esterified d-α-tocopherol acid succinate. Since research prior to '846 invention was directed towards formulating insoluble drugs like paclitaxel using 50% cremophor and 50% dehydrated alcohol, and these formulations precipitates upon dilution with infusion fluid, is unstable on storage and causes untoward adverse reactions, hence the '846 invention was directed towards providing an improved formulation of paclitaxel using water-miscible solubilizers other than cremophor to provide formulations with improved long term stability and safety.

PCT Publication WO 2004/084871 relates to poly(lactic-co-glycolic acid) and poly(lactic acid) (PLA) nanoparticles that encapsulate a low molecular weight and water-soluble drug and deliver the drug to target sites where the particles gradually release the drug over a prolonged period of time. Basically the invention of WO '487 relates to converting a low-molecular weight, water-soluble and non-peptide drug into a hydrophobic drug by interacting it with metal ion and then encapsulating the hydrophobicized drug into PLGA or PLA nanoparticles and allowing a surfactant to be adsorbed onto the surface of the particles. This patent does not relate to or mention anti-cancer drugs like paclitaxel and others and does not provide a composition, which has reduced chemotherapy-induced side-effects.

Research publication published by Fonseca et al, in “Journal of Controlled Release 83 (2002) 273-286” is related to developing a polymeric drug delivery system for paclitaxel, such as paclitaxel loaded poly(lactic-co-glycolic acid) nanoparticles, to be intravenously administered, and which is capable of improving therapeutic index of the drug and is devoid of adverse effects caused due to Cremophor EL. Herein, and in most other prior arts described earlier, the particles obtained are of size anything less than 200 nm. The authors have not provided a composition, which has no free drug and is of a specific defined size range, which has a peculiar surprising advantage, as seen by the inventors described in this present invention.

United States Application No. 20060041019 claims an agent for inhibiting hair loss caused by an antitumor agent wherein the agent is a mixture of cyclic and/or straight chain poly lactic acids having a condensation degree of 3 to 20. Preferably, the mixture of cyclic and/or straight chain poly lactic acids as per the inventors of '019 application is a mixture of polylactic acids that is produced by polymerizing lactide in the presence of the compound represented by formula (3): Me-N(R.sup.1) (R.sup.2) wherein Me represents an alkali metal and R.sup.1 and R.sup.2 each independently represent an aliphatic group or an aromatic group.

It has thus been seen that none of these prior arts have provided a composition and method of manufacturing such compositions of anticancer drugs like paclitaxel, docetaxel and others with substantially reduced alopecia related side-effects. In-spite of the various attempts made earlier to provide anticancer compositions with improved efficacy, none of these compositions show low clinical side effects especially none has provided methods to reduce the specifically distressing side effects of alopecia or hair loss.

There is therefore a need for novel and improved compositions comprising anticancer drugs and methods of treatment using these compositions to overcome the stability problems and to alleviate the various clinical side-effects of the prior known marketed formulations, most importantly reducing the treatment induced alopecia or hair loss and method of preparing the same. There is such a need for example drugs like 5-fluorouracil, doxorubicin, docetaxel, paclitaxel, its derivatives and/or its analogues,

### OBJECTS OF THE INVENTION

The objects of the invention are—
1. To provide novel and improved compositions for cancer therapy having substantially reduced chemotherapy-induced side-effects like alopecia.
2. To provide novel and improved compositions for cancer therapy comprising particles of at least one anticancer drug and at least one polymer, wherein due to the particles being present within a defined particle size range the composition produces substantially reduced chemotherapy-induced side-effects like alopecia.
3. To provide novel and improved compositions for cancer therapy as described above wherein additionally the composition has substantially no free drug; the drug being substantially completely associated with the polymer(s).
4. To provide novel and improved compositions for cancer therapy comprising particles of at least one anticancer drug and at least one polymer; wherein the particles have D10≧80 nm, D50 of about 200 nm and D90≦450 nm; the composition being such that it provides reduced chemotherapy-induced side-effects like alopecia
5. To provide novel and improved compositions for cancer therapy as described in 4 above wherein the particles have D10≧120 nm, D50 of about 200 nm and D90≦350 nm.
6. To provide novel and improved compositions for cancer therapy as described in 5 above wherein the particles have D10≧140 nm, D50 of about 200 nm and D90≦260 nm.
7. To provide novel and improved compositions for cancer therapy as described above wherein the anticancer drug is selected from the group consisting of alkylating agents, antimetabolites, antibiotic anticancer agents, plant alkaloids, anthracenediones, natural products, hormones, hormone antagonists, miscellaneous agents, radiosensitizers, platinum coordination complexes, adrenocortical suppressants, immunosuppressive agent, functional therapeutic agents, gene therapeutic agent, antisense therapeutic agent, tyrosine kinase inhibitor, monoclonal antibody, immunotoxin, radioimmunoconjugate, cancer vaccine, interferon, interleukin, substituted ureas, taxanes and COX-2 inhibitors.
8. To provide novel and improved compositions for cancer therapy as described in 7 above wherein the anticancer drug is preferably chosen from derivatives of taxanes (like paclitaxel, docetaxel), 5-fluorouracil and doxorubicin.
9. To provide novel and improved compositions for cancer therapy as described above wherein the anticancer drug is paclitaxel present in an amount from about 0.5% to about 99.5% by weight of the composition and containing from about 2.0% to about 99.0% by weight of polymer(s).
10. To provide novel and improved compositions for cancer therapy as described above, wherein the polymer is biodegradable polymers like human serum albumin, poly(d,l-lactic-co-glycolic acid) and the like present in an amount ranging from about 2.0% to about 99.0% by weight of the composition.
11. To further provide the above novel and improved compositions for cancer therapy with a secondary polymer selected from the group consisting of temperature and/or pH sensitive polymers like poly(N-acetylacrylamide), poly(N-isopropylacrylamide), poly(N-isopropylacrylamide-co-acrylamide), polyvinylalcohol, polyethyleneglycol, polyacrylamide, poly(methacrylamide) and the like and derivatives thereof.
12. To provide novel and improved composition for cancer therapy as described in 11 above wherein the secondary polymer is poly(N-isopropylacrylamide) used in an amount selected from: the group consisting of from about 0.5% to about 99.0% by weight, from about 1.0% to about 95.0% and from about 2.0% to about 90.0% by weight of the composition.
13. To provide novel and improved compositions for cancer therapy as described in 11 above wherein in presence of the secondary polymer, particles of the composition, upon administration to a mammal, increases in size to about two times its original size in plasma and to about ten times its original size at the tumor site, thus providing targeting and substantially reduced chemotherapy-induced side-effects like alopecia.

14. To provide novel and improved compositions for cancer therapy as described in 1 above wherein the composition comprises paclitaxel in an amount from about 0.5% to about 99.5%, poly(d,l-lactic-co-glycolic acid) in an amount from about 2.0% to about 99.0% and optionally poly(N-isopropylacrylamide) in an amount from about 2.0% to about 90.0%, and one or more pharmaceutically acceptable excipients, carriers or a combination thereof from about 0.01% to about 99.9% by weight of the composition.
15. To provide novel and improved compositions for cancer therapy as described in 1 above wherein the composition comprises paclitaxel in an amount from about 0.5% to about 99.5%, albumin in an amount from about 2.0% to about 99.0% and optionally poly(N-isopropylacrylamide) in an amount from about 2.0% to about 90.0%, and one or more pharmaceutically acceptable excipients, carriers or a combination thereof from about 0.01% to about 99.9% by weight of the composition.
16. To provide a method of making a novel and improved composition as described above comprising the steps of (i) mixing at least one anticancer drug with at least one polymer in a solvent (ii) optionally carrying out step (i) in the presence of one or more pharmaceutically acceptable carriers (iii) obtaining nanoparticles by removing the solvent and (iii) subjecting the nanoparticles to particle sizing (iv) removing any free drug from the composition; the composition being such that it provides substantially reduced chemotherapy-induced side-effects like alopecia.
17. To provide a method of treating a mammal for cancer therapy comprising the step of administering to the mammal a therapeutically effective amount of the said novel and improved compositions comprising particles of at least one anticancer drug and at least one polymer wherein the particles have D10≧80 nm, D50 of about 200 nm and D90≦450 nm and the composition being such that it has substantially no free drug and provides a substantially reduced chemotherapy-induced side-effects like alopecia.
18. To provide a method for reducing chemotherapy-induced side-effects like alopecia of a cancer therapy in a mammal undergoing treatment with anticancer drugs, said method comprising administering a therapeutically effective amount of the said novel and improved compositions comprising particles of at least one anticancer drug and at least one polymer wherein the particles have D10≧80 nm, D50 of about 200 nm and D90≦450 nm and the composition being such that it has substantially no free drug

### SUMMARY OF THE INVENTION

The present invention is directed to novel and improved compositions for cancer therapy having substantially reduced chemotherapy-induced side-effects.

The present invention is directed to novel and improved compositions of anticancer drugs, preferably the poorly soluble anticancer drugs, it's method of manufacturing and methods of treating cancer patients with these compositions having reduced chemotherapy-induced side-effects like alopecia.

The important aspect of the invention is directed towards providing colloidal delivery systems like nanoparticulate compositions of anticancer drugs like taxanes (eg. paclitaxel or docetaxel) and at least one biodegradable polymer such that the composition has a defined particle size range, wherein the particles have D10 greater than or equal to 80 nm, D50 of about 200 nm and D90 less than or equal to 450 nm. Such a defined specific effective particle size range provides a composition which when administered to patients for treatment of cancer therapy, has substantially reduced chemotherapy-induced side-effects like alopecia. The composition is preferably such that it has substantially no free drug; the drug being substantially completely associated with the polymer.

Another aspect of the invention is directed towards providing such a nanoparticulate composition further comprising a secondary polymer, which is temperature and pH sensitive, and optionally other pharmaceutically acceptable carriers, as well as any other desired excipients. Such compositions provides particles which upon administration to a mammal, increases in size about two times its original size in plasma and about ten times its original size at the tumor site, thus providing targeting at the tumor site and substantially reduced chemotherapy-induced side-effects like alopecia.

This invention further discloses a method of making such a nanoparticulate composition comprising the steps of mixing at least one anticancer drug with at least one polymer in the presence of a solvent having optionally one or more pharmaceutically acceptable carriers as well as any desired excipients to provide nanoparticles, removing the solvent and subjecting to particle sizing to obtain particles having a defined particle size like D10≧80 nm, D50 of about 200 nm and D90≦450 nm. The thus obtained nanoparticles of a defined particle size range are further subjected to removal of any free drug. Such a composition when administered to patients provides substantially reduced chemotherapy-induced side-effects like alopecia.

The present invention is thus directed towards providing a method of treatment comprising administering to a mammal in need of a therapeutically effective amount of a nanoparticulate composition according to the invention, which provides substantially reduced chemotherapy-induced side-effects like alopecia. It provides a method for reducing chemotherapy-induced side-effects like alopecia of a cancer therapy in a mammal undergoing treatment with anticancer drugs by administering the said therapeutically effective nanoparticulate composition of the present invention.

Both the foregoing general description and the following detailed description are exemplary and explanatory and are intended to provide further explanation of the invention as claimed. Other objects, advantages, and novel features will be readily apparent to those skilled in the art from the following detailed description of the invention.

### DETAILED DESCRIPTION OF THE INVENTION

The present invention provides novel and improved compositions for cancer therapy.

Many newer anticancer agents are being developed for treating tumors in mammals, but the major disadvantage of anticancer or antitumor agents is that they do not specifically and selectively affect tumor cells; they also affect normal cells and hence produce side-effects.

Attempts are being made in the field of drug delivery to target more and more of these anticancer drugs towards the site of action to improve efficacy and also attempts are being made to provide multiple-drug therapy to enhance effectiveness of anticancer drugs. However, the issue of side-effects is still a major concern, which has not yet been fully addressed, one such major side-effects with chemotherapy being alopecia or hair loss.

Hair loss, or alopecia, is a distressing side-effect for individuals undergoing chemotherapy. Most of the chemotherapy patients experience a great degree of alopecia. Hair regrowth after chemotherapy take from 3 to 6 months, and some percentage of patients fail to experience complete recovery. Chemotherapy-induced alopecia is particularly devastating because it is an outward sign of an otherwise hidden disease, leading some patients to refuse systemic chemotherapy.

Thus in accordance with most preferred aspect of the present invention, there are provided novel and improved compositions for cancer therapy with substantially reduced side-effects. The side-effect preferably being chemotherapy-induced side-effect like alopecia. The composition of the present invention comprises of at least one anticancer drug and at least one polymer.

The anticancer drugs useful in the present invention for cancer therapy are selected from the group consisting of alkylating agents, antimetabolites, antibiotic anticancer agents, plant alkaloids, anthracenediones, natural products, hormones, hormones antagonists, miscellaneous agents, radiosensitizers, platinum coordination complexes, adrenocortical suppressants, immunosuppressive agent, functional therapeutic agents, gene therapeutic agent, antisense therapeutic agent, tyrosine kinase inhibitor, monoclonal antibody, immunotoxin, radioimmunoconjugate, cancer vaccine, interferon, interleukin, substituted ureas, taxanes and COX-2 inhibitors.

The group described above includes: alkylating agents, including: alkyl sulfonates such as busulfan, ethyleneimine derivatives such as thiotepa, nitrogen mustards such as chlorambucil, cyclophosphamide, estramustine, ifosfamide, mechlorethamine, melphalan, and uramustine, nitrosoureas such as carmustine, lomustine, and streptozocin, triazenes such as dacarbazine, procarbazine, and temozolamide, and platinum compounds such as cisplatin, carboplatin, oxaliplatin, satraplatin, and (SP-4-3)-(cis)-amminedichloro-[2-methylpyridine]platinum(II); antimetabolites, including: antifolates such as methotrexate, permetrexed, raltitrexed, and trimetrexate, purine analogs such as cladribine, chlorodeoxyadenosine, clofarabine, fludarabine, mercaptopurine, pentostatin, and thioguanine, pyrimidine analogs such as azacitidine, capecitabine, cytarabine, edatrexate, floxuridine, fluorouracil, gemcitabine, and troxacitabine; natural products, including: antitumor antibiotics such as bleomycin, dactinomycin, mithramycin, mitomycin, mitoxantrone, porfiromycin, and anthracyclines such as daunorubicin (including liposomal daunorubicin), doxorubicin (including liposomal doxorubicin), epirubicin, idarubicin, and valrubicin, enzymes such as L-asparaginase and PEG-L-asparaginase, microtubule polymer stabilizers such as the Taxanes, paclitaxel and docetaxel, mitotic inhibitors such as the vinca alkaloids vinblastine, vincristine, vindesine, and vinorelbine, topisomerase I inhibitors such as the camptothecins, irinotecan and topotecan, and topoisomerase II inhibitors such as amsacrine, etoposide, and teniposide; hormones and hormone antagonists, including: androgens such as fluoxymesterone and testolactone, antiandrogens such as bicalutamide, cyproterone, flutamide, and nilutamide, aromatase inhibitors such as aminoglutethimide, anastrozole, exemestane, formestane, and letrozole, corticosteroids such as dexamethasone and prednisone, estrogens such as diethylstilbestrol, antiestrogens such as fulvestrant, raloxifene, tamoxifen, and toremifene, LHRH agonists and antagonists such as buserelin, goserelin, leuprolide, and triptorelin, progestins such as medroxyprogesterone acetate and megestrol acetate, and thyroid hormones such as levothyroxine and liothyronine; and miscellaneous agents, including altretamine, arsenic trioxide, gallium nitrate, hydroxyurea, levamisole, mitotane, octreotide, procarbazine, suramin, thalidomide, photodynamic compounds such as methoxsalen and sodium porfimer, and proteasome inhibitors such as bortezomib. Molecular targeted therapy agents include: functional therapeutic agents, including: gene therapy agents, antisense therapy agents, tyrosine kinase inhibitors such as erlotinib hydrochloride, gefitinib, imatinib mesylate, and semaxanib, and gene expression modulators such as the retinoids and rexinoids, e.g. adapalene, bexarotene, trans-retinoic acid, 9-cis-retinoic acid, and N-(4-hydroxyphenyl)retinamide; phenotype-directed therapy agents, including: monoclonal antibodies such as alemtuzumab, bevacizumab, cetuximab, ibritumomnab tiuxetan, rituximab, and trastuzumab, immunotoxins such as gemtuzumab ozogamicin, radioimmunoconjugates such as ¹³¹I-tositumomab, and cancer vaccines. Biologic therapy agents include: interferons such as interferon-α₂ₐ and interferon-α_{2b}, and interleukins such as aldesleukin, denileukin diftitox, and oprelvekin. In addition to these agents intended to act against cancer cells, cancer therapies include the use of protective or adjunctive agents, including: cytoprotective agents such as amifostine, dexrazonxane, and mesna, phosphonates such as pamidronate and zoledronic acid, and stimulating factors such as epoetin, darbeopetin, filgrastim, PEG-filgrastim, and sargramostim. Preferably the anticancer drug is a poorly soluble anticancer drug.

The anticancer drug used in the present invention is taxanes and derivatives thereof (e.g. paclitaxel, docetaxel, and derivatives thereof and the like) but does not exclude other anticancer drugs like (for e.g. doxorubicin, methotrexate, cisplatin, daunorubicin, adriamycin, cyclophosphamide, actinomycin, bleomycin, epirubicin, mitomycin, methotrexate, 5-fluorouracil, carboplatin, carmustine (BCNU), methyl-CCNU, cisplatin, etoposide, interferons, camptothecin, phenesterine, tamoxifen, piposulfan, and derivatives thereof and the like). The preferred anti-cancer agent being agents chosen from taxanes, 5-fluorouracil and doxorubicin, the most preferred being taxanes.

The term “taxane” as used herein includes the chemotherapy agents Taxol (generic name: paclitaxel; chemical name: 5.beta.,20-epoxy-1,2a,4,7.beta.,10.beta.,13a-h-exahydroxytax-11-en-9-one, 4,10-diacetate 2-benzoate 13-ester with (2R,3S)—N-benzoyl-3-phenylisoserine) and Taxotere (generic name: docetaxel), second generation Taxanes like Ortataxel and other semi-synthetic derivatives of taxanes. Taxol, an anticancer drug described in the background as well, has a generic name “paclitaxel”, and the registered trade name “Taxol™” (of Bristol-Myers Squibb Company), is a complex polyoxygenated diterpene, originally isolated from the bark of the Pacific yew tree (Taxus brevifolia). It was approved by FDA to treat breast, ovarian, and lung cancers as well as AIDS-related Kaposi's sarcoma. Taxotere-R (Docetaxel), a substance similar to paclitaxel also comes from the needles of the yew tree, is approved by the FDA to treat advanced breast and non-small cell lung cancers that have not responded to other anticancer drugs. Paclitaxel and docetaxel are administered intravenously. But both paclitaxel and docetaxel have side effects that can be serious. Paclitaxel being insoluble in water was formulated in Taxol using Cremophor EL (polyethoxylated castor oil) and ethanol as excipients; which cause serious adverse effects. High incidences of anaphylactic reactions and other hypersensitivity responses were reported with Taxol. Recently a new protein bound nanoparticulate paclitaxel injectable suspension was introduced, brand named Abraxane®, which avoided use of cremophor and was free of solvents, thus being free of cremophor and solvent related adverse effects. But even this composition manifests the other chemotherapy-induced side-effects, one of which is the most traumatic side-effect alopecia or hair loss. Thus, in spite of paclitaxel's good clinical efficacy and it's recognition as one of the biggest advances in oncology medicine, there is still a growing need to provide a paclitaxel composition with much better safety and pharmacokinetic profile in patients avoiding the most traumatic side-effects like alopecia.

The most preferred taxane selected for the present study is paclitaxel though it should be understood that such a study can be extended to other anticancer drugs as well, details of which is provided herewith. Paclitaxel is present in the composition of the present invention in an amount from about 0.5% to about 99.5% by weight, preferably in an amount from about 2.0% to about 95.0% and most preferably in an amount from about 5.0% to about 90.0% by weight of the composition.

The anticancer agents can be used alone or in combination with one or more other agents in the present invention. They may be amorphous, crystalline or mixtures thereof, preferably the agent is substantially amorphous.

The present invention is described herein using several definitions, as set forth herein and throughout the application.

“Pharmaceutically acceptable” as used herein refers to those compounds, materials, compositions, and/or dosage forms which are, within the scope of sound I medical judgment, suitable for use in contact with the tissues of human beings and animals without excessive toxicity, irritation, allergic response, or other problem or complication, commensurate with a reasonable benefit/risk ratio.

“Therapeutically effective amount” refers to an amount that is effective to achieve a desired therapeutic result.

The term “polymer” as used herein refers to a molecule containing a plurality of covalently attached monomer units, and includes branched, dendrimeric and star polymers as well as linear polymers. The term also includes both homopolymers and copolymers, e.g., random copolymers, block copolymers and graft copolymers, as well as uncrosslinked polymers and slightly to moderately to substantially crosslinked polymers.

Term “Biodegradable polymer” means the polymer should degrade by bodily processes to products readily disposable by the body and should not accumulate in the body and the term “biocompatible” describes a substance that does not appreciably alter or affect in any adverse way, the biological system into which it is introduced.

“Poorly soluble” as used herein means the active agent has solubility in water of less than about 10 mg/ml, and preferably, of less than 1 mg/ml at room temperature.

As used herein, “particle size” is used to refer to the size of particles in the composition in diameter, as measured by conventional particle size analyzers well known to those skilled in the art, such as sedimentation field flow fractionation, photon correlation spectroscopy, laser light scattering or dynamic light scattering technology and by using transmission electron microscope (TEM) or scanning electron microscope (SEM). A convenient automated light scattering technique employs a Horiba LA laser light scattering particle size analyzer or similar device. Such analysis typically presents the volume fraction, normalized for frequency, of discrete sizes of particles including primary particles, aggregates and agglomerates. In the present description the particle size characteristics frequently refer to notations of the Dn type, where n is a number from 1 to 99; this notation represents the cumulative distribution of particle size such that n % (by volume basis) of the particles are smaller than or equal to the said size. Typically the particle size is expressed in D10, D50 (median) and D90 values in nm size. The ratio of D90/D10 is a convenient characteristic for identifying the width of the particle size distribution curve. In various aspects of this invention the particle size distribution is narrow, preferably having a ratio of D90/D10 of less than 4, more preferably less than 3 and even more preferably less than 2.0.

As used herein, the term “nm” refers to nanometer, size less than 1 micron, wherein micron is a unit of measure of one one-thousandth of a millimeter.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term. This meaning is applicable to use of the term “about” in context of this application when used to describe % or amount of anticancer drugs, carriers, excipients and others except in case of describing the particle size of the particles of the present invention, wherein, the word “about” refers to a value up to plus or minus 25% of the particular term. This means that D50 of about 200 nm refers to a particle size range of 150 nm to 250 nm.

The term “Chemotherapy-induced side-effects” used herein refers to the unfavorable symptoms generated in mammals due to the administration of anticancer drugs. Examples thereof include hair loss, myelosuppression, vomiting, digestive tract disorders, hepatotoxicity, nephrotoxicity, cerebral toxicity, cardiotoxicity, pulmonary toxicity, stomatitis, dermatopathy, and neurotoxicity. The novel and improved composition according to the present invention is preferably provided for inhibiting or reducing hair loss (or alopecia), among the aforementioned side effects.

“Alopecia” or Hair Loss referred to herein is preferably related to drug induced alopecia, which would damage the hair follicles in the body. It should be understood that the hair follicles on the head have the fastest growth rate and its growth period is long, due to this higher biological activity of the hair organ on the scalp compared to the hair organs at other locations in the body, the hair organ on the scalp is susceptible to anticancer drugs resulting in the damage to the hair matrix cells in the hair follicles. Consequently, the growth of hair matrix cell functions is affected or the hair organ rapidly moves to the resting stage and the hair falls out in the form of atrophic hair.

Earlier attempts for inhibiting hair loss caused due to chemotherapy included, administering combination of anticancer drugs with an antagonist, blocking blood flow to the scalp, intraarterial administration and others, but none of these tasks have provided any significant effect till date. Attempt has been made in the present invention to achieve this task via safe, effective, simple and a novel technique.

A systematic and detailed study of various composition comprising an anticancer drug and at least one polymer in a particulate form revealed a surprising and very useful finding that the physicochemical factors like geometry of the particles plays a very important role in providing a composition for cancer therapy with reduced side-effects like alopecia. It includes particle size, shape, texture, surface characteristics like surface charge, surface hydrophobicity, weight, molecular weight, volume, fraction, any morphology and the like, of which particle size in diameter, one of the most important factors, has been studied in detail in the present invention. When a composition comprising particles of a defined particle size range is administered as a method of treatment to a mammal for cancer therapy, the said composition undergoes selective biodistribution such that it provides more targeting towards site of action and substantially reduced side-effects like alopecia.

Particles in the nanometer size range is reported to be in circulation in the blood when administered and retained in the tumor epithelial cells due to the leaky vasculature reaching the tumor cells but it is also reported in the literature that particles larger than 200 nm diameters are preferentially recognized by reticulo-endothelial systems (RES) of cells and hence is targeted to organs such as the liver, lungs, spleen, lymphatic circulation and the like and removed from the blood circulation. A major part (90%) of the nanosystems injected intravenously generally is lost to the reticulo-endothelial system, mainly fixed macrophages in the liver and spleen after opsonization by proteins present in the blood stream. Thus opsonization or removal of nanoparticulate drug carriers from the body by the mononuclear phagocytic system (MPS), also known as the reticuloendothelial system (RES), is considered as major obstacle in drug targeting. In one article (Current Nanoscience, 2005, 1, 47-64) it is mentioned that particles <100 nm with hydrophilic surfaces undergoes relatively less opsonization and clearance by RES uptake. Hence most of the earlier attempts to make better and effective anticancer compositions have focused on having compositions with particles below 1 micron, preferably below 200 nm or 100 nm to keep the particles in the circulation, avoid being taken up by RES and target towards tumor site. But in most of these prior art compositions the particles are kept at anything below 1 micron, preferably below 200 nm diameter, which may also include particles below about 70 nm in diameter. It was not recognized in any of these earlier attempts that particles below about 70 nm permeates through normal blood capillaries to skin and hair roots and thus such anticancer drug containing particles would cause chemotherapy-induced side-effects like alopecia when used to treat mammals for cancer therapy. Tumor microvasculature is discontinous and highly permeable, and on average, the endothelial pores are 108±32 nm in internal diameter for tumor and are therefore significantly larger and more heterogeneous in size than capillary caveolae whose internal diameter is 58±9 nm. Therefore, the particles above 70 nm may not permeate through normal blood capillary and will significantly reduce the loss of hair.

In the present invention attempt has been successfully made to provide novel and improved compositions for cancer therapy comprising particles of at least one anticancer drug and at least one polymer; wherein the particles have size less than 1 micron in diameter. Preferably the particles have D10≧80 nm, D50 of about 200 nm and D90≦450 nm i.e the particles are of such a size range that 90% of particles have a particle size less than 450 nm and only 10% of particles have a particle size less than 80 nm or lower, with 50% of particles being about 200 nm size. More preferably the particles have D10≧120 nm, D50 of about 200 nm and D90≦350 nm and most preferably the particles have D10≧140 nm, D50 of about 200 nm and D90≦260 nm. It was surprisingly observed that particles up to about 220 nm were not taken up by the reticulo-endothelial system and were available for circulation to be targeted at the tumor site and the particles not being in the size range below 70 nm prevented them from permeating into the hair follicle, thus leading to substantially reduced chemotherapy-induced side-effects like alopecia. The particles of the present invention were surprisingly found to accumulate in tissues other than those containing the RES such as the prostate, pancreas, testes, breast, seminiferous tubules, bone etc. to a significantly greater level and provided reduced alopecia, thus indicating reduced accumulation in sites like skin and hair follicle.

It should be understood that each hair follicle continually goes through three stages: anagen (growth), catagen (involution), and telogen (rest). Anagen is followed by catagen and ultimately the hair follicle enters the telogen stage when the hair shaft matures into a club hair, which is eventually shed from the follicle. At any given point, most of the hair follicles is found in the anagen phase with only a small percentage in the telogen phase and just a few in the catagen phase. Anticancer drugs disrupt this rapidly proliferating bulb matrix cells during the anagen stage. As a result, hair production ceases and the hair shaft become narrower with subsequent breakage and loss of hair. In the present invention, the anticancer drug composition is such that the drug is prevented from permeation into the hair follicle and thus prevents hair loss.

In preferred aspects of the invention the composition comprising the anticancer drug and at least a polymer is a colloidal delivery system, which includes liposomes, microemulsions, micelles, polymer-drug conjugates, nanocapsules, nanospheres, microparticles and nanoparticles, solid-lipid nanoparticles. These delivery systems offer the advantages of targeting, modulation of distribution and flexible formulation and have a polymer structure, which may be designed and produced in a manner that is adapted to the desired objective. The compositions may be delivered by any routes of administration as described herewith like oral, intravenous, subcutaneous, intraperitoneal, intrathecal, intramuscular, intracranial, inhalation, topical, transdermal, rectal, vaginal, intramucosal and the like and may release the drug immediately or release the drug over a period of time by modulating, sustaining, pulsating, delaying or controlling its release from the delivery system by adapting various known methodologies, which is all incorporated within the scope of this invention. The colloidal delivery system may be monolithic wherein the polymer is dispersed along with the drug or it may be coated wherein the polymer is coated on the drug or it encapsulates the drug. Preferred system is nanosystems including nanoparticles and also newer nanosystems that are being developed including nanocages, nanogels, nanofibers, nanoshells, nanorods, nanocontainers etc.

The preferred delivery system is nanoparticulate composition of the anticancer drug which may offer many advantages including, suitable for parenteral administration, can be formulated in a dried form which readily redisperses, provide high redispersibility of the active agent particles present in the nanoparticulate composition, improved targeting at the site of action, increased bioavailability, reduced dosing, improved pharmacokinetic profiles and reduced side-effects. Preferred nanoparticles are sub-micron sized polymeric colloidal particles with the anticancer drug encapsulated within the polymeric matrix or adsorbed or conjugated onto the surface. It also allows controlling the release pattern of drug and sustaining drug levels for a long time by appropriately selecting the polymer materials.

In accordance with embodiments of the present invention, there are provided improved compositions of anticancer drugs wherein the composition is a nanoparticulate composition of the anticancer drug and a polymer as a colloidal delivery system having a particular specific particle size range as defined herewith, the particles being useful for the treatment of primary and metastasized tumors including cancers of prostate, testes, breast, lung, kidney, pancreas, bone, spleen, liver, brain and the like and others with a significantly reduced side-effects especially the chemotherapy-induced-alopecia. Preferably the composition comprises of at least one anticancer drug from about 0.5% to about 99.5% by weight and at least one polymer from about 2.0% to about 99.0% by weight of the composition. In preferred embodiments the anticancer drug is paclitaxel presented as nanoparticulated composition comprising at least a polymer in an amount ranging from about 2.0% to about 99.0% by weight of the composition.

Biodegradable polymers used in the present invention are inclusive of natural, synthetic and semi-synthetic materials.

Examples of natural polymers include proteins, peptides, polypeptides, oligopeptides, polynucleic acids, polysaccharides (e.g., starch, cellulose, dextrans, alginates, chitosan, pectin, hyaluronic acid, and the like), fatty acids, fatty acid esters, glycerides, fats, lipids, phospholipids, proteoglycans, lipoproteins, and so on, and their modifications. Proteins include albumins, immunoglobulins, caseins, insulins, hemoglobins, lysozymes, a-2-macroglobulin, fibronectins, vitronectins, fibrinogens, lipases, and the like. Proteins, peptides, enzymes, antibodies and combinations thereof, can also be used as stabilizers in the present invention if required to improve stabilization. Preferred protein is albumin preferably used in an amount from about 2.0% to 99.0% by weight, more preferably 5.0% to 95.0% and most preferably from about 10.0% to about 90.0% by weight of the composition.

Synthetic polymers include polyaminoacids like gelatin, polyvinyl alcohol, polyacrylic acid, polyvinyl acetate, polyesters, polyacrylates, polyvinyl pyrrolidone, polyethoxyzoline, polyacrylamide, polyvinyl pyrrolidinone, polyalkylene glycols, polylactides, polyglycolides, polycaprolactones, or copolymers thereof, and the like, and suitable combinations of any two or more thereof, especially ∝-hydroxycarboxylic acids, polyhydroxyethyl methacrylate, poly(ε-caprolactone), poly(β-hydroxybutyrate), poly(hydroxyvalerate) and (β-hydroxybutyrate-hydroxyvalerate) copolymers, polymalic acid, polylactic acid), poly(glycolic acid), poly(d,l-lactic-co-glycolic acid), amphiphilic block polymers of polylactic acid-polyethylene oxide, polyalkylene glycol, polyethylene oxides, block copolymers of polyethylene oxide-polypropylene oxide, polyanhydrides, polyorthoesters, polyphosphazanes, pullulan.

Preferably delivery systems of the invention use biodegradable/biocompatible polymers to encapsulate the anticancer drug. These biodegradable primary polymers may be those which release the immediately on administration or those which delay the release of the anticancer active agent and maintain the nanoparticulate composition in the target site for a longer period of time for therapeutic effectiveness. Preferred primary polymer is poly(d,l-lactic-co-glycolic acid) or PLGA, which is a biodegradable polymer, permitted in the formulation of modified release galenic preparations. PLGA is a hydrophobic copolymer, the degradation of which, caused by a hydrolysis reaction, gives rise to two normal biological substrates, lactic acid and glycolic acid, which are metabolized at the end of aerobic glycolysis to CO2 and H2O. The rate of biodegradation of PLGA depends on the respective proportions of lactic acid and glycolic acid, 50:50 ratio being a preferred ratio. PLGA is completely biocompatible and causes a moderate foreign body reaction. PLGA used in the present invention is preferably in an amount from about 2.0% to 99.0% by weight, more preferably 5.0% to 95.0% and most preferably from about 10.0% to about 90.0% by weight of the composition.

According to another aspect of this invention, it includes, targeting the anticancer drug towards the site of action by various techniques, this includes amongst other techniques, conjugation of targeting ligands to drugs or drug containing nanoparticulated compositions to direct them to their target sites, or coating/associating the composition with temperature and/or pH sensitive polymers.

According to this above described aspect in order to achieve targeted release of the active ingredient at the tumor site, a temperature sensitive and outer surface modified nanoparticles are prepared by applying a temperature responsive interpolymer complex capable of showing thermal responsiveness in an aqueous solution like poly(N-acetylacrylamide), poly(N-isopropylacrylamide), poly(N-isopropylacrylamide-co-acrylamide), polyvinylalcohol, polyethyleneglycol, polyacrylamide, poly(methacrylamide), to the nanoparticles encapsulating the anticancer drug like paclitaxel. Such nanoparticles with hydrophilic surfaces would circulate in the blood for longer period of time and because of the thermal sensitivity of the particles i.e showing upper critical solution temperature (UCST) or lower critical solution temperature (LCST) in an aqueous solution, the particle size increases when injected in-vivo at 37° C.; the particle size further increases several folds when the particles are accumulated in tumor due to difference in physiological conditions in tumor microenvironment and the encapsulated active drug is released at the tumor site. PH sensitive polymers that can be used include polyacrylates, cellulose acetate phthalates and the like.

The drug encapsulated nanoparticles in the present invention are engineered in such a way that under in-vitro conditions, at room temperature; the particles have D10≧80 nm, D50 of about 200 nm and D90≦450 nm, preferably have D10≧120 nm, D50 of about 200 nm and D90≦350 nm and more preferably have D10≧140 nm, D50 of about 200 nm and D90≦260 nm but interestingly due to the temperature sensitiveness of the particles, when these particles are injected in-vivo, the particle size increases to about two times its original size in plasma. Thus even if during scale-up and commercial manufacturing, few of the particles of the composition comprising the drug and polymer may not achieve to fall in the defined particle size range, in-vivo the particles would always be in the range of particle size, which is prevented from permeation from normal blood capillaries to skin and hence to hair roots and would remain in circulation in blood for longer period of time to be finally targeted at the site of action. When these particles reach the tumor, they increase in size to about ten times its original size at the tumor site and are also permeated through leaky and hyperpermeable tumor microvasculature where the particles are retained (i.e enhanced permeation and retention effect) and the drug released. This ultimately leads to reduction in alopecia when such compositions are administered to treat various types of cancer. The compositions having almost nil free drug in it, which has an added advantage in reducing the alopecia related side-effects. The preferred secondary polymer used in the composition of the present invention is temperature and/or pH sensitive polymer like poly(N-isopropylacrylamide), used in an amount from about 0.5% to about 99.0%, preferably from about 1.0% to about 95.0% and most preferably from about 2.0% to about 90.0% by weight of the said composition.

Thus in accordance with preferred embodiments of the present invention there are provided methods for preparing such temperature sensitive and outer surface modified nanoparticles encapsulating anticancer drug like paclitaxel for immediate or controlled and site specific-delivery at the tumor site, thus providing maximum therapeutic effect of the drug with minimum adverse effects at a lower dose of the active ingredient.

Pharmaceutical compositions of anti-cancer drugs like paclitaxel according to this invention include the nanoparticulated compositions described above comprising the drug and pharmaceutically acceptable carriers thereof. Suitable pharmaceutically acceptable carriers are well known to those skilled in the art. These include non-toxic physiologically acceptable carriers, excipients or adjuvants or vehicles for parenteral injection, for oral administration in solid or liquid form, for rectal administration, nasal administration, intramuscular administration, subcutaneous administration and the like. Preferably the composition is parenteral injection composition administered as IV bolus injections or by subcutaneous or intramuscular route.

Compositions suitable for parenteral injection may comprise physiologically acceptable sterile aqueous or nonaqueous dispersions, suspensions or emulsions and sterile powders for reconstitution into sterile injectable dispersions or suspensions. Examples of suitable aqueous and nonaqueous carriers, diluents, solvents, or vehicles include water, aliphatic or aromatic alcohols like absolute ethanol, octanol, alkyl or aryl halides like dichloromethane, ketones like acetone, aliphatic, cycloaliphatic, or aromatic hydrocarbons like hexane, cyclohexane, toluene, benzene, and polyols (propyleneglycol, polyethylene-glycol, glycerol, and the like), N-hydroxy succinimide, carbodiimide, suitable mixtures thereof, vegetable oils (e.g. soybean oil, mineral oil, corn oil, rapeseed oil, coconut oil, olive oil, safflower oil, cotton seed oil and the like) and injectable organic esters such as ethyl oleate, alkyl, aryl or cyclic ethers like diethyl ether, tetrahydrofuran, acetonitrile and aqueous buffered solutions, chloroform and the like. Proper fluidity can be maintained, for example, by the use of a coating such as lecithin, by the maintenance of the required particle size in the case of dispersions or suspensions, and by the use of surfactants.

The nanoparticulate pharmaceutical compositions may also contain in addition to active agents and solvents, excipients or adjuvants such as preserving, wetting, emulsifying, surface stabilizers, surface active agents and dispensing agents, all of which examples is known in the art and is included within the scope of this invention. Prevention of the growth of microorganisms can be ensured by various antibacterial and antifungal agents, such as parabens, chlorobutanol, phenol, sorbic acid, and the like wherever applicable. It may also be desirable to include isotonic agents, such as sugars, sodium chloride, and the like, buffering agents like phosphate, pthalate, acetate, citrate, borate and the like.

The nanoparticulate compositions of the invention can be sterile filtered or manufactured in sterile conditions at every stage of manufacturing. This obviates the need for heat sterilization, which can harm or degrade an active agent, as well as result in crystal growth and particle aggregation of the active agent. The composition as a colloidal delivery system may be finally presented as lyophilized powder or as suspension, suspended in a biocompatible aqueous liquid. The biocompatible liquid may be selected from water, buffered aqueous media, saline, buffered saline, buffered solutions of aminoacids, proteins, sugars, carbohydrates, vitamins or synthetic polymers, lipid emulsions and the like.

In an important aspect of this invention there is provided nanoparticles encapsulating anticancer drug like paclitaxel, its derivatives or its analogs and methods of manufacturing nanoparticles encapsulating paclitaxel, its derivatives or analogs to achieve maximum encapsulation efficiency, such that the nanoparticulated composition has substantially no free drug in it. It is thus the object of this invention to provide a method of fractionating the nanoparticles encapsulating paclitaxel, its derivatives or analogs to a specific defined particle size range and provide a method of subjecting the nanoparticles to a process to remove any free drug in the composition, most of all the drug being associated with the polymer, such that the composition when administered to mammals for treatment produces substantially reduced side-effects like alopecia or hair loss.

The compositions according to the present invention, which includes microparticles, liposomes, nanocapsules, nanospheres, and nanoparticles and others described earlier are manufactured by the standard conventional methods used in the art but with an additional step of fractionating the particles to a defined particle size range as desired and subjecting the particles to a treatment to remove all the free drug not encapsulated or associated with the polymer, the same has been exemplified in detail in the embodiments described herewith. The process for making the nanoparticulate pharmaceutical compositions of the present invention encompasses all techniques to make microparticluate/nanoparticulate compositions. In a preferred aspect of the invention, the process comprises the steps of dissolving and/or dispersing the drug and polymer(s) in aqueous solution and/or solvents or mixture of solvents, mixing the two solutions under stirring to form the emulsion or precipitation, optionally mixing in presence of additional pharmaceutically acceptable carriers or excipients, homogenizing the same under low or high pressure to obtain nanoparticles of a desired particle size, removing the solvent by any technique, one of it being use of reduced pressure, subjecting the nanoparticles to particle sizing if required to obtain the defined particle size range of the present invention, ultrafiltering the nanosuspension through 30 kilodalton membrane to remove all the free drug and finally lyophilizing in vials and storing till further studies.

A method of treating a mammal in accordance with this invention comprises the step of administering to the mammal in need of treatment an effective amount of the said novel and improved compositions of the above-described anticancer drugs and polymers, which would provide substantially, reduced chemotherapy-induced alopecia.

Thus in accordance with a particularly preferred aspect of the present invention, there is provided method for reducing chemotherapy-induced side-effects like alopecia of a cancer therapy in a mammal undergoing treatment with anticancer agents, said method comprising administering a therapeutically effective amount of the said novel and improved compositions comprising particles of at least one anticancer drug and at least one polymer as described herein. The composition being such that it has particles within a defined particle size range as described in the invention herewith and has substantially no free drug in it.

### EXAMPLES

### Example 1

### Synthesis of PLGA Nanoparticles Encapsulating Paclitaxel

The nanoparticles from poly(d,l-lactic-co-glycolic acid) (PLGA) were synthesized using double emulsion approach via w/o/w double emulsion. In a typical experiment, 100 mg of PLGA was dissolved in 2 mL dichloromethane and 10 mg paclitaxel was dissolved in 1.0 mL of absolute ethanol. Both solutions were slowly mixed together with stirring. A primary water-in-oil (w/o) emulsion was made by emulsifying 500 μL phosphate buffer saline in above solution. The primary water-in-oil emulsion was then further emulsified in poly(N-acetylacrylamide) solution to form the water-in-oil-in-water (w/o/w emulsion). The w/o/w emulsion thus made was homogenized to form the paclitaxel-loaded nanoparticles on evaporation of the solvents. The solution was then centrifuged and the nanoparticles in the desired size range were selectively separated. The nanoparticles were then dispersed in sterile water and lyophilized immediately for future use.

### Example 2

### PLGA Coupled Covalently to Pullulan Micellar Nanometer Aggregates and Loading of Paclitaxel

PLGA was coupled covalently to pullulan by activating PLGA with N-hydroxy succinimide. The pullulan-PLGA complex was purified using gel filtration and characterized by FTIR, H-NMR and mass spectroscopy. The hydrophobized pullulan solution was lyophilized and kept in deep freeze for future use.

100 mg of hydrophobized pullulan was dissolved in 10 mL of water and the solution vortexed to form the micelles. A paclitaxel solution prepared in ethanol was added slowly to the micellar solution and dissolved until the solution was clear indicative of drug encapsulation in micellar formulation. Drug loaded particles in the desired range were preferentially separated and the solution was lyophilized.

The encapsulation efficiency or loading capacity and the release behaviour of paclitaxel from the nanoparticles were determined by standard techniques using HPLC and particle size determined using the conventional particle size analyzer.

Coating of Nanoparticles with Thermosensitive Polymers:

Drug loaded nanoparticles were suspended in aqueous buffer (pH 4-5). To this solution, a solution of carbodi-imide was added and the resulting solution was vortexed and continuously stirred at room temperature for 4 hours. The nanoparticles were then separated by centrifugation (or by filtration or dialysis). An aqueous solution of the polymer poly(N-acetylacrylamide) was added dropwise to the nanoparticles suspension and the mixture was vortexed. The solution was then further stirred, the particles purified and lyophilized for future use.

### Fractionation of Nanoparticles in a Particular Size Range:

10.0 mg lyophilized powder of paclitaxel-loaded nanoparticles was suspended in aqueous buffer with the aid of sonication. The solution was filtered through 0.2 μm Millipore filtration unit and the filtrate was subjected to asymmetrical flow field-flow fractionation using the manufacturer's standard protocol for fractionation of particles using this technique. Different fractions were collected and subjected to particle size analysis using the standard techniques to determine the particle size and size distribution.

### Example 3

### Preparation of Paclitaxel—Human Serum Albumin Nanoparticles

1800 mg human serum albumin was dissolved in sterile water for injection. 200 mg of paclitaxel was separately dissolved in ethanol. The ethanolic solution was added slowly under high speed stirring to the aqueous solution of human serum albumin. The emulsion formed was passed through high-pressure homogenizer for a time sufficient to obtain desired size of nanoparticles. Ethanol was removed from the nanoparticles under reduced pressure after which it was subjected to particle sizing by first passing it through 0.2 micron followed by 0.1 micron filter. Frantionated nanoparticles were sterile filtered through 0.2 micron filter, ultrafiltered and lyophilized in vials. Particles were tested for various parameters.

TABLE 1

| Sr. no. | Test | Results |
|---|---|---|
| 1 | Paclitaxel content | 1 mg/10 mg of lyophilized powder. |
| 2 | pH of suspension | 6.8 |
| 3 | Free drug content | Nil |
| 4 | Cumulative volume | D10 - 70.8 nm |
| | distribution of nanoparticles | D50 - 97.9 nm |
| | | D90 - 99.8 nm |

### Example 4

### Preparation of Paclitaxel—Human Serum Albumin Nanoparticles

675 mg human serum albumin was dissolved in sterile water for injection. 75 mg of paclitaxel was separately dissolved in ethanol. The ethanolic solution was added under stiffing to the aqueous solution of human serum albumin. The emulsion formed was passed through homogenizer at a low pressure for a time sufficient to obtain desired size of nanoparticles. Ethanol was removed from the nanoparticles under reduced pressure after which it was ultrafiltered through 30 kiloDalton membrane to remove free drug and then lyophilized in vials. Obtained particles were tested for various parameters.

TABLE 2

| Sr. no. | Test | Results |
|---|---|---|
| 1 | Paclitaxel content | 1 mg/10 mg of lyophilized powder. |
| 2 | pH of suspension | 6.8 |
| 3 | Free drug content | Nil |
| 4 | Cumulative volume | D10 - 143.4 nm |
| | distribution of nanoparticles | D50 - 178.5 nm |
| | | D90 - 285.9 nm |

### Example 5

### Preparation of Paclitaxel-Human Serum Albumin Nanoparticles with LCST Polymer

1800 mg of human serum albumin and 200 mg of poly(N-isopropylacrylamide) (a LCST polymer) was dissolved in sterile water for injection. 200 mg paclitaxel was separately dissolved in ethanol. Further steps followed were similar to that given in Example 3 above.

Particles obtained in the experiments with LCST polymer were fractionated to obtain particles of a desired range. In one such experiment the obtained particles were studied for particle size changes at various temperature conditions, results of which is given in Table 3 below as an example to demonstrate increase in particle size with increase in temperature.

TABLE 3

| | Temperature | | | | |
|---|---|---|---|---|---|
| | 25° C. | 30° C. | 35° C. | 37° C. | 38° C. |
| Average | 90.0 nm | 92.8 nm | 98 nm | 130 nm | <1000 nm |
| Particle size | | | | | |

The results show that the particles comprising paclitaxel and albumin, in the presence of a secondary polymer like LCST polymer, when subjected to various temperature conditions, demonstrate an increase in particle size, at a temperature of 37 degree (eg. plasma temperature), the particles increase in size to about two times its original size and at a temperature of 38 degree (eg. tumor temperature) the particles increase in size to about ten times its original size.

### Example 6

### Preparation of PLGA-Paclitaxel—LCST Polymer Nanoparticles

Paclitaxel and poly(d,l-lactic-co-glycolic acid) (PLGA) was dissolved in acetone. poly(N-isopropylacrylamide) was dissolved in water for injection, followed by addition of polyvinyl alcohol to this aqueous phase. The paclitaxel-PLGA solution was added to the aqueous phase slowly under stirring. Acetone was removed from this emulsion under reduced pressure. The nanoparticles thus obtained were subjected to particles sizing, removal of free drug process and lyophilization respectively.

### Example 7

### Preparation of Paclitaxel—PLGA-Human Serum Albumin Nanoparticles

900 mg human serum albumin was dissolved in sterile water for injection. 100 mg each of paclitaxel and PLGA were separately dissolved in chloroform. The paclitaxel-PLGA solution was added under stirring to the aqueous solution of human serum albumin under high speed mixing to form the O/W emulsion. The emulsion formed was passed through homogenizer at low pressure for a time sufficient to obtain desired size of nanoparticles. Residual ethanol was removed from the nanoparticles under reduced pressure after which it was ultrafiltered through 30 kiloDalton membrane to remove free drug and lyophilized. Obtained particles were tested for various parameters.

TABLE 2

| Sr. no. | Test | Results |
|---|---|---|
| 1 | Paclitaxel content | 1 mg/10 mg of lyophilized powder. |
| 2 | pH of suspension | 6.9 |
| 3 | Free drug content | Nil |
| 4 | Cumulative volume | D10 - 176.6 nm |
| | distribution of nanoparticles | D50 - 233.8 nm |
| | | D90 - 318.4 nm |

### Example 8

### Effect of Chemotherapy on Hair Growth Pattern in Mice

Seven weeks old male BALB/c mice used for the study were housed in cages and allowed free access to food and water. They were maintained under standard condition (25° C. room temperature, 12 hr light and 12 hr dark cycle).

Samples injected for the study were—Reference: (commercially available albumin bound paclitaxel injectable suspension) Test: (sample obtained from Example 3) and Control: Saline (Vehicle).

A murine model was developed to study chemotherapy-induced alopecia. Under anesthesia, telogen mice that had gone through several postnatal hair cycles were induced to enter anagen by depilation of all telogen hair shafts. This was performed by using electric hair clipper followed by use of commercially available depilation cream to the back skin. By using this technique, all depilated telogen hair follicles immediately began to transform into anagen follicles (stages Ito VI) (refer Paus et al., American Journal of Pathology, 144, 719-734 (1994). The above steps were performed to induce a highly synchronized anagen development phase in the mice as opposed to a spontaneous anagen development phase. At anagen VI phase (9th day after depilation), Test and Reference samples (20 mg/kg) and equivalent amount of Control were administered intravenously to three groups of mice having four mice each for the study.

After the treatment all animals were observed visually for hair growth and sign of alopecia and digitally photographed for records. Scoring of hair growth pattern, at different time intervals after the chemotherapy and vehicle treatments, was done based on the hair growth index described below:

Score for hair growth index
0=No hair growth
1=Mild hair growth with severe alopecia.
2=Moderate hair growth with scattered alopecia.
3=Good and uniform hair growth with no sign of alopecia.

Hair growth score index for each treatment is given in the table 4 below.

TABLE 4

Hair growth score
index (Mean ± SEM)

| Groups | Day 1 | Day 10 |
|---|---|---|
| Control | 0.00 | 3.00 ± 0.00 |
| (Saline i.v.) | | |
| Test | 0.00 | 2.66 ± 0.33 |
| (20 mg/kg i.v.) | | |
| Reference | 0.00 | 2.0 ± 0.57 |
| (20 mg/kg i.v.) | | |

Higher hair growth score index indicates better hair growth.

The above data indicates that Test treated mice showed better hair growth in comparison to the Reference and has a value closer to the control.

### Example 9

### Effect of Chemotherapy on Hair Growth Pattern in Mice

The same study as described above was done with another Test sample obtained from Example 4.

Samples injected for the study were—Reference: (commercially available albumin bound paclitaxel injectable suspension) Test: (sample obtained from Example 4) and Control: Saline (Vehicle).

Hair growth score index for each treatment in this study is given in the table 5 below.

TABLE 5

Hair growth score
index (Mean ± SEM)

| Groups | Day 1 | Day 10 |
|---|---|---|
| Control | 0.00 | 2.25 ± 0.25 |
| (Saline i.v.) | | |
| Test | 0.00 | 2.00 ± 0.00 |
| (20 mg/kg i.v.) | | |
| Reference | 0.00 | 1.50 ± 0.28 |
| (20 mg/kg i.v.) | | |

The above data indicates that Test treated mice showed much better hair growth in comparison to the Reference and has a score much closer to the Control samples, the Reference treated mice having the lowest hair growth score index. There is a statistically significant difference in the hair growth index scores of Control and Reference (p<0.05; t=1.964, df=6, n=4). This data further indicate that the Test sample showed reduced chemotherapy-induced side-effects like alopecia.

### Example 10

### Studies in Tumor Bearing Mice

Samples taken for this study were: (a) Reference (commercially available albumin bound paclitaxel injectable suspension) (b) Test I (Sample obtained from example 4) (c) Test II (Sample obtained from example 5)

The purpose of this experiment was to study tumor retentiveness and leakiness behavior of the nanoparticles of the present invention (Test samples) in comparison to the Reference sample. Tumor bearing ICRC mice (carrying spontaneous mammary tumor) were taken, the mice were divided into three groups (n=5) based on average tumor size and dosed (0.06 mg/100 mm³) with Reference and Test samples, through intratumor route. After a fixed time interval of 8 his the mice were sacrificed, tumor and plasma were harvested and subjected to analysis for paclitaxel.

The tumor plasma ratio of paclitaxel in test and reference samples was calculated and was found to be 71.61 in example 4, and 355.7 in example 5 and 19.96 in Reference. This data indicates that paclitaxel was retained 3.58 times with Test sample of example 4 and 17.80 times with Test sample of example 5 in comparison to the Reference. This further indicates less leakiness of test samples in comparison to reference and support reduced side effects like alopecia as seen in Test sample of example 4. Test sample with additional temperature sensitive polymers in the composition as exemplified in example 5 provided much better retentiveness due to swelling of the particles to a particle size as defined in the invention and hence have much lesser leakiness, which may result in substantially reduced side-effects like alopecia.

1. Novel and improved compositions for cancer therapy having substantially reduced chemotherapy-induced side-effects like alopecia.

2. Novel and improved compositions for cancer therapy according to claim 1 comprising particles of at least one anticancer drug and at least one polymer.

3. Novel and improved compositions for cancer therapy according to claim 2 wherein the particles are present within a defined particle size range; the composition being such that it produces substantially reduced chemotherapy-induced side-effects like alopecia.

4. Novel and improved compositions for cancer therapy according to claim 2 wherein the particles have D10≧80 nm, D50 of about 200 nm and D90≦450 nm.

5. Novel and improved compositions for cancer therapy according to claim 2 wherein the particles have D10≧120 nm, D50 of about 200 nm and D90≦350 nm.

6. Novel and improved compositions for cancer therapy according to claim 2 wherein the particles have D10≧140 nm, D50 of about 200 nm and D90≦260 nm.

7. Novel and improved compositions for cancer therapy according to claim 2 wherein said composition has substantially no free drug and wherein said drug is substantially completely associated with the polymer(s)

8. Novel and improved compositions for cancer therapy according to claim 2 wherein said particles have a particle size distribution ratio of D90/D10 less than 4.0.

9. Novel and improved compositions for cancer therapy according to claim 2 wherein said particles have a particle size distribution ratio of D90/D10 less than 3.0.

10. Novel and improved compositions for cancer therapy according to claim 2 wherein said particles have a particle size distribution ratio of D90/D10 less than 2.0.

11. Novel and improved compositions for cancer therapy according to claim 2 wherein the anticancer drug is selected from the group consisting of alkylating agents, antimetabolites, antibiotic anticancer agents, plant alkaloids, anthracenediones, natural products, hormones, hormones antagonists, miscellaneous agents, radiosensitizers, platinum coordination complexes, adrenocortical suppressants, immunosuppressive agent, functional therapeutic agents, gene therapeutic agent, antisense therapeutic agent, tyrosine kinase inhibitor, monoclonal antibody, immunotoxin, radioimmunoconjugate, cancer vaccine, interferon, interleukin, substituted ureas, taxanes and COX-2 inhibitors.

12. Novel and improved compositions for cancer therapy according to claim 11 wherein the anticancer drug is one or more of chlormethine, chlorambucile, busulfan, thiotepa, chlorambucil, cyclophosphamide, estramustine, ifosfamide, mechlorethamine, melphalan, uramustine, carmustine, lomustine, streptozocin, dacarbazine, procarbazine, temozolamide, cisplatin, carboplatin, oxaliplatin, satraplatin, (SP-4-3)-(cis)-amminedichloro-[2-methylpyridine}-platinum(II), methotrexate, permetrexed, raltitrexed, trimetrexate, cladribine, chlorodeoxyadenosine, clofarabine, fludarabine, mercaptopurine, pentostatin, thioguanine, azacitidine, capecitabine, cytarabine, edatrexate, floxuridine, 5-fluorouracil, genicitabine, troxacitabine, bleomycin, dactinomycin, adriamycin, actinomycin, mithramycin, mitomycin, mitoxantrone, porfiromycin, daunorubicin, doxorubicin, liposomal doxorubicin, epirubicin, idarubicin, vairubicin, phenesterine, tamoxifen, piposulfancamptothesin, L-asparaginase, PEG-L-asparaginase, paclitaxel, docetaxel, taxotere, vinblastine, vincristine, vindesine, vinorelbine, irinotecan, topotecan, amsacrine, etoposide, teniposide, fluoxymesterone, testolactone, bicalutamide, cyproterone, flutamide, nilutamide, aminoglutethimide, anastrozole, exemestane, formestane, letrozole, dexamethasone, prednisone, diethylstilbestrol, fulvestrant, raloxifene, tamoxifen, toremifine, buserelin, goserelin, leuprolide, triptorelin, medroxyprogesterone acetate, megestrol acetate, levothyroxine, liothyronine, altretamine, arsenic trioxide, gallium nitrate, hydroxyurea, levamisole, mitotane, octreotide, procarbazine, suramin, thalidomide, methoxsalen, sodium porfimer, bortezomib, erlotinib hydrochloride, gefitinib, imatinib mesylate, semaxanib, adapalene, bexarotene, trans-retinoic acid, 9-cis-retinoic acid, and N-(4-hydroxyphenyl)retinamide, alemtuzumab, bevacizumab, cetuximab, ibritumomab tiuxetan, rituximab, trastuzumab, gemtuzumab ozogamicin, tositumomab, interferon-α2a, interferon-α2b, aldesleukin, denileukin diftitox, and oprelvekin and derivatives thereof and the like.

13. Novel and improved compositions for cancer therapy according to claim 12 wherein the anticancer drug is selected from taxanes, doxorubicin, and 5-fluorouracil.

14. Novel and improved compositions for cancer therapy according to claim 13 wherein the anticancer drug is taxanes and derivatives thereof.

15. Novel and improved compositions for cancer therapy according to claim 14 wherein the taxane is chosen from paclitaxel and docetaxel.

16. Novel and improved compositions for cancer therapy according to claim 2 wherein the polymer is biodegradable polymer.

17. Novel and improved compositions for cancer therapy according to claim 16 wherein the biodegradable polymer is selected from a group comprising proteins, peptides, fatty acids, lipids, phospholipids, polynucleic acid, polysaccharides, proteoglycans, lipoproteins, ∝-hydroxycarboxylic acids, poly(ε-caprolactone), poly(β-hydroxybutyrate), poly(hydroxyvalerate) and (β-hydroxybutyrate-hydroxyvalerate) copolymers, polymalic acid, poly(lactic acid), poly(glycolic acid), poly(d,l-lactic-co-glycolic acid), amphiphilic block polymers of polylactic acid-polyethylene oxide, polyalkylene glycol, polyethylene oxides, block copolymers of polyethylene oxide-polypropylene oxide, polyanhydrides, polyorthoesters, polyphosphazanes, pullulan, poly(N-vinyl pyrrolidone), polyvinyl alcohol, polyvinyl acetate, polyesters, polyaminoacids, polyacrylates, polyvinyl pyrrolidone, polyethoxyzoline and other synthetic and natural polymers or co-polymers thereof and the like and derivatives and mixtures thereof.

18. Novel and improved compositions for cancer therapy according to claim 17 wherein said protein is albumin.

19. Novel and improved compositions for cancer therapy according to claim 2 wherein said composition comprise from about 0.5% to about 99.5% by weight of said anticancer drug and from about 2.0% to about 99.0% by weight of said polymer(s).

20. Novel and improved compositions for cancer therapy according to claim 2 wherein the anticancer drug is paclitaxel and polymer is poly(d,l-lactic-co-glycolic acid).

21. Novel and improved compositions for cancer therapy according to claim 2 wherein the anticancer drug is paclitaxel and polymer is albumin.

22. Novel and improved compositions for cancer therapy according to claim 2 further comprising a secondary polymer.

23. Novel and improved compositions for cancer therapy according to claim 22 wherein the secondary polymer is selected from the group consisting of temperature and pH sensitive polymers.

24. Novel and improved compositions for cancer therapy according to claim 23 wherein the temperature and/or pH sensitive polymers is selected from the group consisting of poly(N-acetylacrylamide), poly(N-isopropylacrylamide), poly(N-isopropylacrylamide-co-acrylamide), polyvinylalcohol, polyethyleneglycol, polyacrylamide, poly(methacrylamide) and the like and derivatives thereof.

25. Novel and improved compositions for cancer therapy according to claim 24 wherein said temperature and/or pH sensitive polymer is poly(N-isopropylacrylamide).

26. Novel and improved compositions for cancer therapy according to claim 19 further comprising secondary polymer in an amount from about 0.5% to about 99.0%, from about 1.0% to about 95.0% and from about 2.0% to about 90.0% by weight of the said composition.

27. Novel and improved compositions for cancer therapy according to claim 22 wherein in presence of the secondary polymer, particles of the composition, upon administration to a mammal, increases in size to about two times its original size in plasma and to about ten times its original size at the tumor site, thus providing targeting and substantially reduced chemotherapy-induced side-effects like alopecia.

28. Novel and improved compositions for cancer therapy according to claim 2 wherein said composition is a colloidal delivery system.

29. Novel and improved compositions for cancer therapy according to claim 28 wherein the colloidal delivery system is lyophillized.

30. Novel and improved compositions for cancer therapy according to claim 28 wherein the colloidal delivery system is such that the particles are suspended in a biocompatible aqueous liquid.

31. Novel and improved compositions for cancer therapy according to claim 1 wherein the composition comprises paclitaxel in an amount from about 0.5% to about 99.5%, poly(d,l-lactic-co-glycolic acid) in an amount from about 2.0% to about 99.0% and optionally poly(N-isopropylacrylamide) in an amount from about 2.0% to about 90.0%, and one or more pharmaceutically acceptable excipients, carriers or a combination thereof from about 0.01% to about 99.9% by weight of the composition.

32. Novel and improved compositions for cancer therapy according to claim 1 wherein the composition comprises paclitaxel in an amount from about 0.5% to about 99.5%, albumin in an amount from about 2.0% to about 99.0% and optionally poly(N-isopropylacrylamide) in an amount from about 2.0% to about 90.0%, and one or more pharmaceutically acceptable excipients, carriers or a combination thereof from about 0.01% to about 99.9% by weight of the composition.

33. A method of making a novel and improved composition for cancer therapy according to claim 1 comprising the steps of (i) mixing at least one anticancer drug with at least one polymer in a solvent (ii) optionally carrying out step (i) in the presence of one or more pharmaceutically acceptable carriers (iii) obtaining nanoparticles by removing the solvent and (iii) subjecting the nanoparticles to particle sizing such that the obtained particles have D10≧80 nm, D50 of about 200 nm and D90≦450 nm and have substantially no free drug; the composition being such that it provides substantially reduced chemotherapy-induced side-effects like alopecia.

34. A method of treating a mammal for cancer therapy comprising the step of administering to the mammal a therapeutically effective amount of the said novel and improved compositions comprising particles of at least one anticancer drug and at least one polymer wherein the particles have D10≧80 nm, D50 of about 200 nm and D90≦450 nm and the composition being such that it has substantially no free drug and provides a substantially reduced chemotherapy-induced side-effects like alopecia.

35. A method for reducing chemotherapy-induced side-effects like alopecia of a cancer therapy in a mammal undergoing treatment with anticancer drugs, said method comprising administering a therapeutically effective amount of the said novel and improved compositions comprising particles of at least one anticancer drug and at least one polymer wherein the particles have D10≧80 nm, D50 of about 200 nm and D90≦450 nm and the composition being such that it has substantially no free drug.
